(19) United States
(12) Patent Application Publication
(10) Pub. No.: US 2007/0038175 A1
(43) Pub. Date: Feb. 15, 2007

(54) Enhanced needleless medication delivery system

(76) Inventor: Kurt Daniel Van Laar, Simi Valley, CA (US)

Correspondence Address: KURT D. VAN LAAR, 2624 CITRONELLA COURT, SIMI VALLEY, CA, 93063, US

(21) Appl. No.: 11/196,588

(22) Filed: Aug. 4, 2005

(51) Int. Cl. A61M 5/30

(52) U.S. Cl. 604/68

(57) ABSTRACT

A Needleless injector patch for the intra-muscular, subcutaneous, or intra-dermal delivery of a fluid medicament to a patient includes a plurality of cylindrical members “Micro-Injectors” which have a closed end and an sealed orifice end, the Micro-Injectors contain a pyrotechnic gas generating charge, a diaphragm or a piston assembly and a quantity of medication. An onboard microprocessor programmatically selects the time to initiate a Micro-Injector unit by applying current to the pyrotechnic charge generating a volume of gas which pushes a diaphragm or piston pressurizing the medication to the point that a rupture element bursts allowing the medication to be expelled as a fine stream at high pressure that pierces the epidermis to a controlled depth delivering the medication as an injection.

### STATEMENT OF FEDERALLY SPONSORED RESEARCH

There is NO Federal Sponsorship.

### FIELD OF INVENTION

This invention relates to Needleless Injection of medical products more specifically a transdermal like patch that delivers pharmaceuticals via a high speed Micro Injection Stream “Micro-Ject”.

### BENEFITS OF INVENTION

There are several benefits to using this invention: 1. Many of the pharmacological materials have molecules that are too large to be dosed via the transdermal route but are injectable. 2. Medications that can not be used transdermally and may cause unpleasant reactions when used orally or may require buffering or are not suitable for oral delivery, can benefit from this invention because it mitigates the problems of oral ingestion by a needleless injection. 3. Because of the multi-shot capability of the invention and it's integrated microprocessor a controlled and programmatic delivery regimen is possible.

### BACKGROUND OF THE INVENTION

Currently transdermal patches are limited to small molecules that are compatible with a specific set of solvents that are capable of passing through the epidermis and carrying the medication, all in a non-toxic mode. There are several needleless injection systems currently on the market all of which are too large to be used as a patch. The needleless units use compressed gas, springs, a solenoid pump or in one design pyrotechnics. These designs are not conducive to the creation of a controllable medication patch.

### BRIEF SUMMARY OF THE INVENTION

The invention utilizes the rapid gas generation of an enclosed gas generator to push a piston in a bore or in the preferred embodiment deforming a diaphragm, causing the pressurization of a controlled quantity of a fluid medication causing it to rupture a rupture film and be expelled through a orifice creating a needle like stream that passes through the skin and into the cutaneous, subcutaneous or intramuscular layers depending upon the quantity of the gas generating pyrotechnics used.

Further the actuation of the gas generating elements are under microprocessor or micro computer control.

### DESCRIPTION OF RELATED ART

U.S. patent application Ser. No. 11/090,883 Van Laar, This patent is follow on enhancement of U.S. patent application Ser. No. 11/090,883 in that the piston assembly has been replaced with a diaphragm and a porous ceramic disk as the preferred embodiment.

6,800,070 Mazidji, et al. Are using a needle to do the injection. By contrast this invention 1. Eliminates the needle, 2. Can be multi shot, 3. The patch is much smaller than a bracelet but could be incorporated into a bracelet or used in conjunction with a super glue, see claims 8 and 9

6,730,028 Eppstein et al. have used pyrotechnic charges to create holes in a biological membrane to facilitate transdermal applications. This is a update based on 6,352,506 In both cases the skin is ablated to allow medications to flow through a damaged or disrupted epidermis. This invention injects the medication programmatically to a selectable depth i.e. subcutaneous or intra-muscular on a regulated basis.

In 6,352,506 Eppstein et al. have used pyrotechnic charges to create holes in a biological membrane to facilitate transdermal applications.

4,089,334 Schwebel, et al Provide pyrotechnically powered needleless injector that is a mechanical device that uses a firing pin and cap mechanism to ignite the pyrotechnic charge. In contrast this patent 1. Eliminates the large mechanical device with a plurality of miniature injectors. 2. Manages the Micro-Ject pyrotechnic charges programmatically and 3. can be worn for an extended period.

### BRIEF DESCRIPTION OF THE SEVERAL VIEWS OF THE DRAWINGS

Elements

10. Pyrotechnic Gas Generator

15. Gas Generator Space

20. Gas retaining strip, glass and silicon fiber reinforced silicon adhesive

30. Polymer strip containing injection barrels

40. Piston

50. Rupture element “strip”, “Membrane” or “disk”

60. Injection orifices

70. Medication containing volume

80. Medication Injector “Micro-Ject” Assembly

90. Patch

100. Microprocessor and associated electronics

110. Ground Plane

120. Super Adhesive Layer, Die Cut

130. Control and programming connector

135. Energy Storage Super Cap or Battery

140. Sealing layer of adhesive backed Teflon tape

150. Peal off release paper

160. Die Cut hole for Injection [larger than Injection orifices]

170. Squib connection leads [screen printed, copper PCB or Wires]

180. Injector Barrel

200. Flexible diaphragm

210. Injector Barrel Bottom with domed cavity

220. Porous Ceramic Disk

230. Ceramic Spacer

240. Injector Barrel Top Assembly

FIG. 1. This view shows the top surface of the patch with the outline of the patch [140], the Control and programming connector [130], and a dotted outline of where the medication Injection Assemblies are located [80].

FIG. 2. Depicts a section view through layer [30] showing a plurality of Micro-Ject assemblies[80] and the microprocessor[100] embedded in a fixable polymer such as but not limited to silicon rubber.

FIG. 3. An enlargement and sectioned view of the preferred embodiment of the Micro-Ject assembly wherein [210] Injector Barrel Bottom with domed cavity contains the Injectable materials. The rupture diaphragm[50] seals the barrel bottom and bursts at a preset pressure. A separating flexible diaphragm of silicon or equivalent material[200] serves as the sealing membrane between the gag generator cavity and the injectable. The porous ceramic disk[220] serves to prevent hot materials from the gas generator from contacting the diaphragm[200]. A spacer made of ceramic or equivalent materials. The Injector barrel top assembly [240] contains the gas generator, provides the pressure on the diaphragm[200] to seal the injectables cavity. On ignition of the gas generator the volume above the diaphragm pressurize causing the injectables to be pressurized until the rupture diaphragm[50] bursts and the injectables are expelled through the injection orifice[60] and into the target patent.

FIG. 4. An enlargement and sectioned view of an alternate embodiment of the patch, through an injector assembly[80] important elements are: the squibs[10], the pistons[40] that compresses the injectables[70] which is expelled via orifice[60] after sufficient pressure has been generated to rupture the rupture strip[50], the medication is formed into a fine stream by the orifice and passes unobstructed through the hole[160] in the adhesive layer[120]. The Gas retaining strip[20] acts as the top seal of the Injector barrel[ 180]

FIG. 5. A simplified schematic diagram showing the programming and charging connector[ 130] the power storage device[135] that is ether a battery or super cap, The microprocessor[100] several squibs[10] and ground[ 110]

FIG. 6. A schematic view of the patch[90] showing a plurality of Micro-Ject injector assemblies [80], and the controlling microprocessor [100]

FIG. 7. This is a cross-section of the patch depicting the layers that comprise the patch. The peal off release liner[ 150] is used to protect the adhesive layer[120] that is die cut to provide passages [160 FIG. 3] through the adhesive to facilitate the jet of injectable material.

FIG. 8. This depicts the electronics where [100] is the Microprocessor and related electronics, the gas generating squibs are [10], Micro-Injector barrel assemblies[80], [110] is the ground plane, [170] are the control wiring between the squibs and the microprocessor

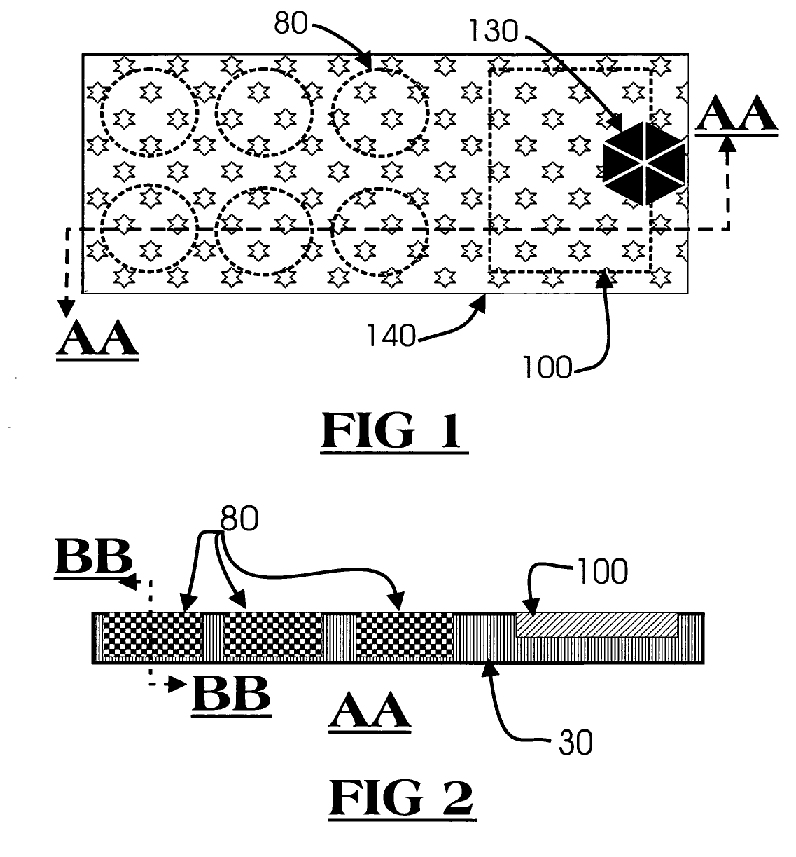

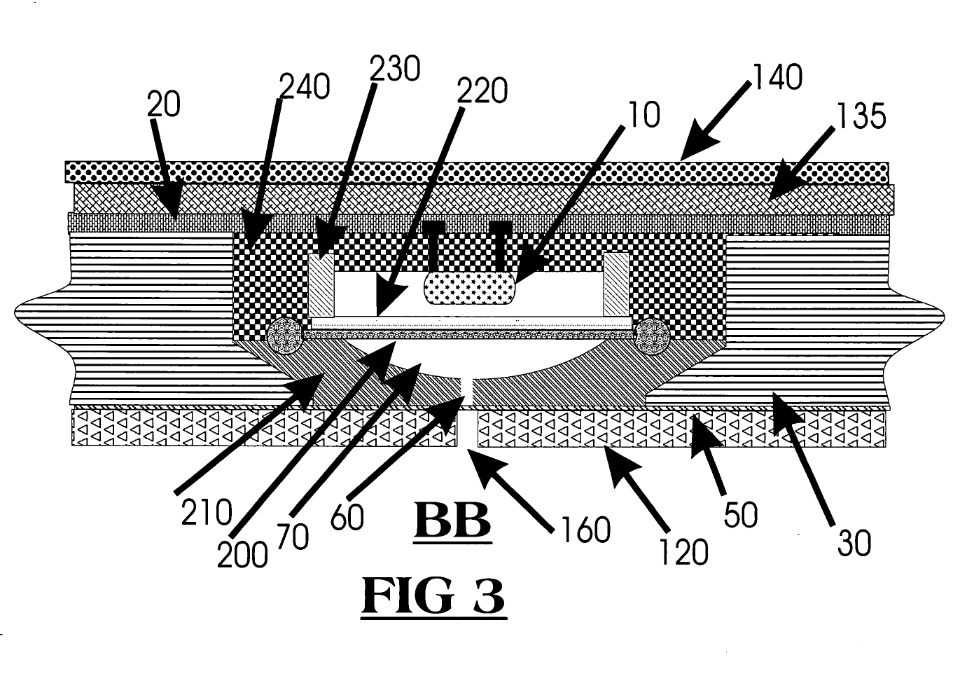

### DETAILED DESCRIPTION OF THE INVENTION

How it Works

The patch is placed in an accompanying programmer to set the data and time of each injection. The release tape[150] is removed and the patch is placed on the patents skin. At pre-selected times the microprocessor's program will select a injector and apply a charge of electricity to a Gas Generator[ 10]. The pyrotechnic element rapidly burns generating gasses that cause the Gas Generator chamber[15] to pressurize. In the Proffered Embodiment:

The pressurized gas causes the Diaphragm[200] to deform transferring the pressure to the injectables until the burst pressure of the rupture element[50] is reached at which time the injectables are expelled as a narrow stream and at high velocity through the orfice[60] that penetrates the patients skin delivering the medication subcutaneously or intramuscularly depending on the pyrotechnic charge and the rupture diaphragm selections.

In the Alternate Embodiment:

pressurized gasses force the piston[40] down on the preloaded medication[70]. When the pressure on the medication chamber reaches the rupture pressure of the sealing rupture element[50], the element ruptures allowing the medication to exit at high speed through an orifice[60] creating a fine stream at high pressure that penetrates the patients skin delivering the medication subcutaneously or intramuscularly depending on the pyrotechnic charge and the rupture diaphragm selections.

What is claimed:

1. A Needleless Injection where in:
a. The appearance is that of a traditional transdermal being approximately 12 mm to 40 mm wide by from 80 mm to 150 mm long with a height of less than 10 mm-15 mm, and;
b. There are a plurality of Micro-Ject injectors, and;
c. Each of the Micro-Ject injectors preferably use a pyrotechnic charge a Sodium Azide compound[10] to generate a gas volume to deform a diaphragm[200] compressing the injectable content of the medication volume [70], and;
d. The compressed medication volume causes the rupture element[50] to rupture at a pre-selected pressure allowing the compressed medications[70] to be forced through an orifice[60] creating a stream of the compressed material of such a diameter and velocity that it penetrates the subject's epidermis to a controlled depth, and;
e. The scheduling of the initiating of each Micro-Ject injector is programmatically controlled by software resident in the microprocessor [100], and;
f. The delivery depth is controlled by orifice diameter and pyrotechnic charge volume and the viscosity of the medication, where delivery is selectable from dermal to subcutaneous.

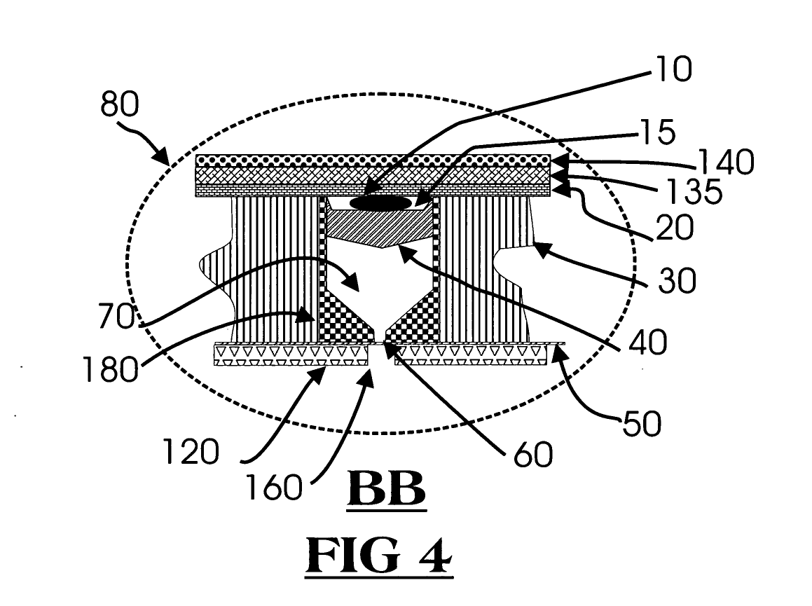

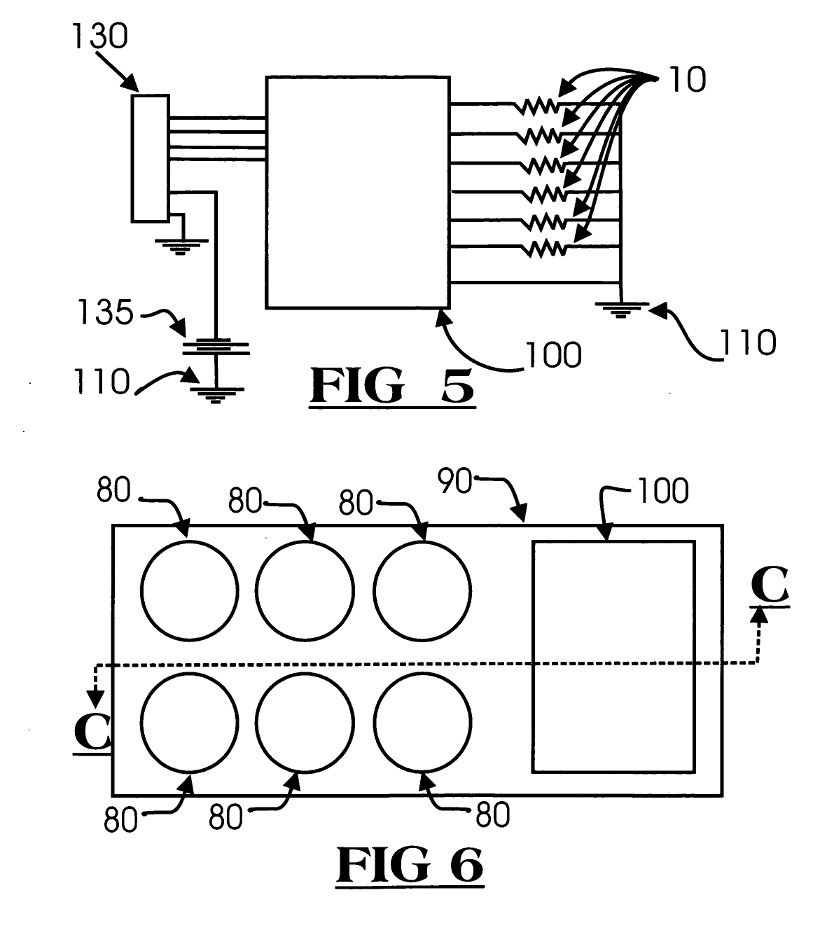

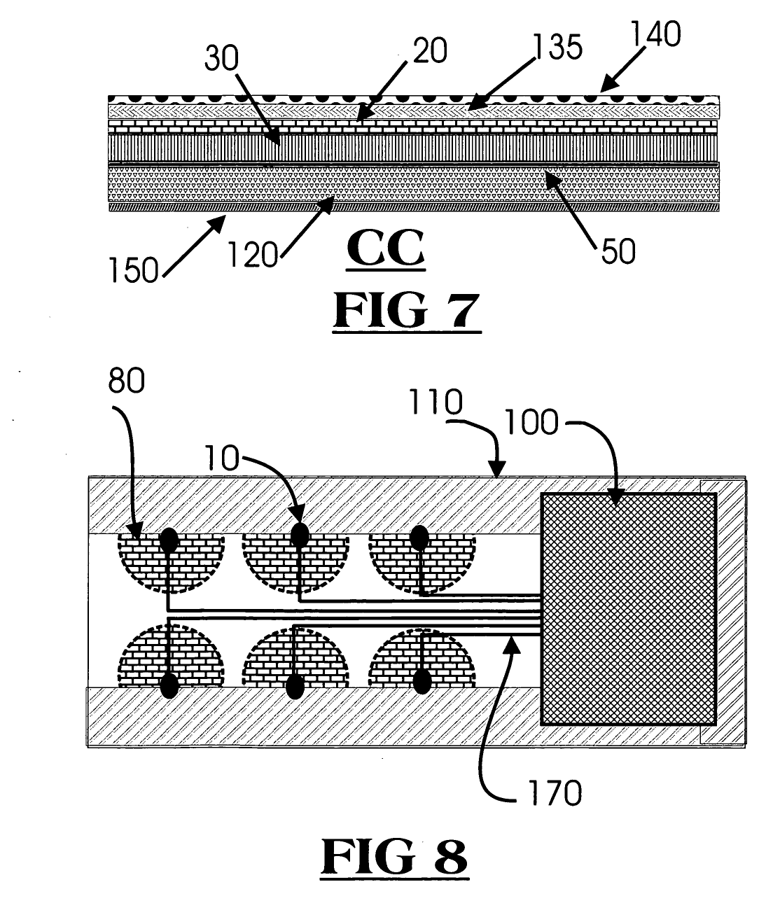

2. An Needleless Injection Patch as described in claim 1. Wherein the Barrel Assembly[80] is configured using a Piston as depicted in FIG. 4

3. An Needleless Injection Patch as described in claims 1 or 2. Wherein the pyrotechnic material is Sodium Azide and shaped Double-Base Smokeless Powders wherein the geometry is used to provide a controlled output pressure throughout the injection cycle.

4. An Needleless Injection Patch as described in claims 1 or 2. Wherein the pyrotechnic material is A mixture of urazole with Potassium Perchlorate in a stoichiometric ratio.

5. An Needleless Injection Patch as described in claims 1 or 2. Wherein the pyrotechnic material is A mixture of urazole with Potassium Perchlorate in a stoichiometric ratio and shaped Double-Base Smokeless Powders wherein the geometry is used to provide a controlled output pressure throughout the injection cycle.

6. An Needleless Injection Patch as described in claims 1 or 2. Wherein the injectables may differ from Micro-Ject to Micro-Ject i.e. Micro-Ject A, B and C may contain compound one, and Micro-Jects D, E and F may contain compound two and so on.

7. An Needleless Injection Patch as described in claims 1 or 2. Wherein the adhesives will adhere to skin such that it would be extremely painful to remove without the use of a solvent.

8. An Needleless Injection Patch as described in claims 1 or 2. Wherein the injectables are medications.

9. An Needleless Injection Patch as described in claim 1. Wherein the injectables are control or incapaciting agents and the microprocessor can be commanded to release the agent based on onboard sensors.

10. An Needleless Injection Patch as described in claims 1 or 2. Wherein the injectables are control or incapaciting agents and the microprocessor can be commanded to release the agent based on a remote command.

11. An Needleless Injection Patch as described in claims 1 or 2. Wherein the injectables are pain management agents and the microprocessor can be commanded to release the agent based on a remote command.

12. An Needleless Injection Patch as described in claims 1 or 2. Wherein the injectables are pain management agents and the microprocessor can be commanded to release the agent based on onboard sensors.

13. An Needleless Injection Patch as described in claims 1 or 2. Wherein the injectables are diabetes management medications and the microprocessor can be commanded to release the agent based on onboard sensors.

14. An Needleless Injection Patch as described in claims 1 or 2. Wherein the there is a sensor to detect removal or tamper and indicates such activity and disables the patch,
